(19) United States
(12) Patent Application Publication
(10) Pub. No.: US 2017/0319840 A1
(43) Pub. Date: Nov. 9, 2017

(54) USING ULTRASOUND SHEAR-WAVES TO ENHANCE SKIN PERMEABILITY

(71) Applicant: Elwha LLC, Bellevue, WA (US)

(72) Inventors: JESSE R. CHEATHAM, III, SEATTLE, WA (US); JOEL CHERKIS, REDMOND, WA (US); PAUL H. DIETZ, REDMOND, WA (US); TOM DRISCOLL, SAN DIEGO, CA (US); WILLIAM GATES, MEDINA, WA (US); RODERICK A. HYDE, REDMOND, WA (US); MURIEL Y. ISHIKAWA, LIVERMORE, CA (US); NEIL JORDAN, REDMOND, WA (US); JORDIN T. KARE, SAN JOSE, CA (US); ERIC C. LEUTHARDT, ST. LOUIS, MO (US); NATHAN P. MYHRVOLD, MEDINA, WA (US); PATRICK NEILL, SAMMAMISH, WA (US); TONY S. PAN, BELLEVUE, WA (US); ROBERT C. PETROSKI, SEATTLE, WA (US); DAVID R. SMITH, DURHAM, NC (US); ELIZABETH A. SWEENEY, SEATTLE, WA (US); DESNY S. TAN, KIRKLAND, WA (US); CLARENCE T. TEGREENE, MERCER ISLAND, WA (US); DAVID LAWRENCE TENNENHOUSE, HILLSBOROUGH, CA (US); YAROSLAV A. URZHUMOV, BELLEVUE, WA (US); GARY WACHOWICZ, LAKE TAPPS, WA (US); LOWELL L. WOOD, JR., BELLEVUE, WA (US); VICTORIA Y.H. WOOD, LIVERMORE, CA (US)

(73) Assignee: Elwha LLC

(21) Appl. No.: 15/150,086

(22) Filed: May 9, 2016

(51) Int. Cl. A61M 37/00; A61F 13/15; A61M 1/00

(52) U.S. Cl. CPC A61M 37/0092; A61M 1/0023; A61F 13/15; A61M 2205/3303; A61M 2037/0007

(57) ABSTRACT

Described embodiments include a system, method, and apparatus. A system includes an extracellular-fluid collection device configured to be positioned at a location on a skin of a mammal. In an embodiment, the mammal includes a live human. The system includes an ultrasonic wave transmitter configured to emit ultrasonic shear waves directable at the location. The ultrasonic shear waves have a frequency or amplitude selected to increase a permeability of the skin of the mammal to an extracellular-fluid. In an embodiment, the system includes a sensor configured to determine a rate or amount of extracellular-fluid collected by the extracellular-fluid collection device. In an embodiment, the system includes a fluid collection controller configured to regulate a parameter of ultrasonic shear waves transmitted by the ultrasonic wave transmitter in response to a determined rate or amount of fluid collected by the extracellular-fluid collection device.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### Priority Applications

NONE

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

For example, and without limitation, an embodiment of the subject matter described herein includes a system. The system includes an extracellular-fluid collection device configured to be positioned at a location on a skin of the mammal. In an embodiment, the mammal includes a live human. The system includes an ultrasonic wave transmitter configured to emit ultrasonic shear waves directable at the location. The ultrasonic shear waves have a frequency or amplitude selected to increase a permeability of the skin of the mammal to an extracellular-fluid. In an embodiment, the emitted ultrasonic shear waves may be directable at the location by aligning, aiming, or positioning the ultrasonic wave transmitter such that a significant portion of the emitted ultrasonic shear waves affect the location on the skin. For example, directable include capable of being directed at the location. For example, directable include able to be directed at the location. For example, directable includes the ultrasonic shear waves being guided or steerable at the location.

In an embodiment, the system includes a housing carrying the extracellular-fluid collection device and the ultrasonic wave transmitter. In an embodiment, the system includes a sensor configured to determine a rate or amount of extracellular-fluid collected by the extracellular-fluid collection device. In an embodiment, the system includes a fluid collection controller configured to regulate a parameter of ultrasonic shear waves transmitted by the ultrasonic wave transmitter in response to a determined rate or amount of fluid collected by the extracellular-fluid collection device. In an embodiment, the system includes a cavitation sensor configured to detect a cavitation event in the mammal. In an embodiment, the system includes a cavitation controller configured to limit a power of the ultrasonic shear waves directed at the location to a level below a cavitation threshold of the mammal.

For example, and without limitation, an embodiment of the subject matter described herein includes a system. The system includes an extracellular-fluid collection device configured to be positioned at a location on a skin of a mammal. The system includes an ultrasonic wave transmitter configured to emit ultrasonic shear waves directable at the location. The ultrasonic shear waves having a frequency or amplitude selected to increase a permeability of the skin of the mammal to an extracellular-fluid. The system includes a sensor configured to determine a rate or amount of extracellular-fluid collected by the extracellular-fluid collection device. The system includes a fluid collection controller configured regulate a parameter of ultrasonic shear waves transmitted by the ultrasonic wave transmitter in response to the determined rate or amount of extracellular-fluid collected by the extracellular-fluid collection device.

For example, and without limitation, an embodiment of the subject matter described herein includes a system. The system includes an extracellular-fluid collection device configured to be positioned at a location on a skin of a mammal. The system includes an ultrasonic wave transmitter configured to emit ultrasonic shear waves directable at the location. The ultrasonic shear waves having a frequency or amplitude selected to increase a permeability of the skin of the mammal to an extracellular-fluid. The system includes a cavitation sensor configured to determine a cavitation event in the mammal to the emitted ultrasonic shear waves. The system includes a cavitation controller configured to limit a power of the ultrasonic shear waves directed at the location to a level below a cavitation threshold of the mammal.

For example, and without limitation, an embodiment of the subject matter described herein includes a method. The method includes positioning an extracellular-fluid collection device at a location on a skin of a mammal. The method includes positioning an ultrasonic wave transmitter proximate to the location on the skin and orientated to direct ultrasonic shear waves emitted by the transmitter at the location. The method includes applying an ultrasonic shear wave excitation to the location on the skin. The ultrasonic shear wave excitation having a frequency or amplitude selected to increase a permeability of the skin to an extracellular-fluid. The method includes determining a rate or amount of extracellular-fluid collected by the extracellular-fluid collection device.

In an embodiment, the method includes regulating a parameter of ultrasonic shear waves transmitted by the ultrasonic wave transmitter in response to the determined rate or amount of extracellular fluid collected by the extracellular-fluid collection device. In an embodiment, the method includes detecting a cavitation event in the mammal. In an embodiment, the method includes limiting a power of the ultrasonic shear waves directed at the location to a level below a cavitation threshold of the mammal. In an embodiment, the method includes creating a vacuum between extracellular-fluid collection device and the location on the skin.

For example, and without limitation, an embodiment of the subject matter described herein includes a system. The system includes means for collecting an extracellular-fluid at a location on a skin of a mammal. The system includes means for transmitting ultrasound shear waves at the location. The ultrasonic shear waves having a frequency or amplitude selected to increase a permeability of the skin of the mammal to an extracellular-fluid.

In an embodiment, the system includes means for determining a rate or amount of extracellular-fluid collected by the extracellular-fluid collection device. In an embodiment, the system includes means for regulating a parameter of ultrasonic shear waves transmitted by the ultrasonic wave transmitter in response to the determined rate or amount of fluid collected by the extracellular-fluid collection device. In an embodiment, the system includes means for detecting a cavitation event in the mammal. In an embodiment, the system includes means for limiting a power of the ultrasonic shear waves directed at the location to a level below a cavitation threshold of the mammal.

The foregoing summary is illustrative only and is not intended to be in any way limiting. In addition to the illustrative aspects, embodiments, and features described above, further aspects, embodiments, and features will become apparent by reference to the drawings and the following detailed description.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example environment 100 in which embodiments may be implemented;

FIG. 2 illustrates an example operational flow 200 in which embodiments may be implemented;

FIG. 3 illustrates an example system 300 in which embodiments may be implemented;

FIG. 4 illustrates an example environment 400 in which embodiments may be implemented;

FIG. 5 illustrates an example operational flow 500 in which embodiments may be implemented; and

FIG. 6 illustrates an example system 600 in which embodiments may be implemented.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

This application makes reference to technologies described more fully in United States Patent Application No. To be assigned, USING ULTRASOUND SHEAR-WAVES TO ENHANCE SKIN PERMEABILITY, naming Jesse R. Cheatham III et al. as inventors, filed on May 9, 2016, is related to the present application. That application is incorporated by reference herein, including any subject matter included by reference in that application.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various implementations by which processes and/or systems and/or other technologies described herein can be effected (e.g., hardware, software, and/or firmware), and that the preferred implementation will vary with the context in which the processes and/or systems and/or other technologies are deployed. For example, if an implementer determines that speed and accuracy are paramount, the implementer may opt for a mainly hardware and/or firmware implementation; alternatively, if flexibility is paramount, the implementer may opt for a mainly software implementation; or, yet again alternatively, the implementer may opt for some combination of hardware, software, and/or firmware. Hence, there are several possible implementations by which the processes and/or devices and/or other technologies described herein may be effected, none of which is inherently superior to the other in that any implementation to be utilized is a choice dependent upon the context in which the implementation will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to implement an operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described below. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, module, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

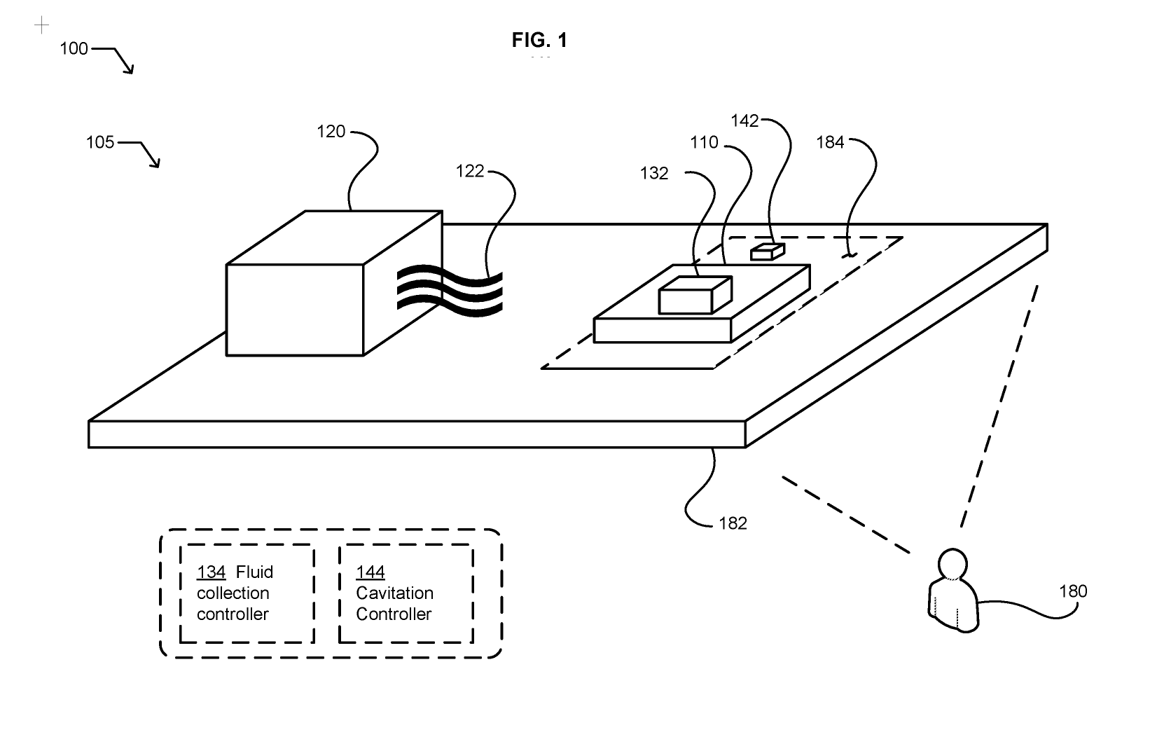

FIG. 1 illustrates an example environment 100 in which embodiments may be implemented. The environment includes a mammal 180 having a skin 182. The environment includes a system 105. The system includes an extracellular-fluid collection device 110 configured to be positioned at a location 184 on the skin of the mammal. In an embodiment, the mammal includes a live mammal. In an embodiment, the mammal includes a live human. The system includes an ultrasonic wave transmitter 120 configured to emit ultrasonic shear waves 122 directable at the location. The ultrasonic shear waves have a frequency or amplitude selected to increase a permeability of the skin of the mammal to an extracellular-fluid. In an embodiment, the ultrasonic shear waves have a clinically relevant frequency or amplitude selected to increase a permeability of the skin of the mammal to an extracellular-fluid. In an embodiment, the emitted ultrasonic shear waves may be directable at the location by aligning, aiming, or positioning the ultrasonic wave transmitter such that a significant portion of the emitted ultrasonic shear waves affect the location on the skin. For example, directable includes capable of being directed at the location. For example, directable includes able to be directed at the location. For example, directable includes the ultrasonic shear waves being guided or steerable at the location. In an embodiment, the ultrasonic shear waves have a frequency or amplitude selected to create a permeability in the skin of the mammal to an extracellular-fluid. In an embodiment, the ultrasonic shear waves have a clinically relevant frequency or amplitude selected to increase a permeability of the skin of the mammal to an extracellular-fluid.

In an embodiment, the extracellular-fluid collection device 110 is configured to be removably attached to the skin 182 of the mammal 180. In an embodiment, the extracellular-fluid collection device includes an ultrasound-transparent extracellular-fluid collection device. For example, in an embodiment, the extracellular-fluid collection device may have greater than a 90% transparency to the emitted ultrasonic shear waves 122. In an embodiment, the extracellular-fluid collection device includes a transdermal patch. In an embodiment, the extracellular-fluid collection device includes an absorbent extracellular-fluid collection device. In an embodiment, the extracellular-fluid collection device includes a strap or band configured to attach the extracellular-fluid collection device to the mammal. In an embodiment, the extracellular-fluid collection device includes an extracellular-fluid collection device having a handheld form factor. In an embodiment, the extracellular-fluid collection device includes a collecting chamber.

In an embodiment, the ultrasonic wave transmitter 120 is configured to emit traveling ultrasonic shear waves 122 directable at the location 184 on the skin 182. In an embodiment, the ultrasonic wave transmitter is configured to emit ultrasonic shear waves focused at the location on the skin. In an embodiment, the ultrasonic wave transmitter is configured to emit ultrasonic standing waves at the location on the skin. In an embodiment, the ultrasonic wave transmitter is configured to emit both ultrasonic shear waves and longitudinal waves directed at the location on the skin. In an embodiment, the ultrasonic wave transmitter is configured to emit surface shear waves. In an embodiment, the ultrasonic wave transmitter is configured to emit two-dimensional surface shear waves. For example, two-dimensional surface shear waves includes shear waves generally limited to a skin depth, or otherwise relatively shallow in the skin. In an embodiment, the ultrasonic wave transmitter is configured to emit three-dimensional shear waves. For example, three-dimensional shear waves may generally be expected to penetrate deeper in the tissue of the mammal than two-dimensional surface shear waves.

In an embodiment, the ultrasonic shear waves 122 have a frequency and amplitude selected to increase a permeability of the skin 182 of the mammal 180 to an extracellular-fluid. In an embodiment, the shear ultrasonic shear waves have a frequency greater than 1 MHz. In an embodiment, the shear ultrasonic shear waves have a frequency greater than 2 MHz. In an embodiment, the shear ultrasonic shear waves have a frequency greater than 10 MHz. In an embodiment, the shear ultrasonic shear waves have a frequency greater than 16 MHz.

In an embodiment, the ultrasonic wave transmitter 120 includes a handheld form factor. In an embodiment, the ultrasonic wave transmitter is configured to be removeably attached to the skin 182.

In an embodiment, the system 105 includes a housing carrying the extracellular-fluid collection device 110 and the ultrasonic wave transmitter 120. In an embodiment, the housing includes a flexible housing. In an embodiment, the housing includes a structure carrying the extracellular-fluid collection device and the ultrasonic wave transmitter. In an embodiment, the housing includes a housing incorporating the extracellular-fluid collection device and the ultrasonic wave transmitter. In an embodiment, the system includes a sensor 132 configured to determine a rate or amount of extracellular-fluid collected by the extracellular-fluid collection device 110. In an embodiment, the system includes a fluid collection controller 134 configured to regulate a parameter of ultrasonic shear waves transmitted by the ultrasonic wave transmitter in response to a determined rate or amount of fluid collected by the extracellular-fluid collection device. For example, a parameter of the ultrasonic shear waves may include a frequency of the ultrasonic shear waves. For example, a parameter of the ultrasonic shear waves may include an amplitude of the ultrasonic shear waves. For example, an amplitude of the ultrasonic shear waves may include an intensity or power level. For example, an amplitude of the ultrasonic shear waves may include a pulse-width modulation or pulse-duration modulation. For example, a parameter of the ultrasonic shear waves may include a waveform of the ultrasonic shear waves.

In an embodiment, the system 105 includes a cavitation sensor 142 configured to detect a cavitation event in the mammal 180. In an embodiment, the cavitation sensor is configured to detect a cavitation event in the mammal associated with the emitted ultrasonic shear waves 122. In an embodiment, the cavitation sensor is configured to detect a vibrational signature associated with a cavitation event in the mammal. In an embodiment, the cavitation sensor includes a sensor ultrasonic wave receiver configured to detect a cavitation event in the mammal in response to sensor ultrasound waves received by the cavitation sensor. In an embodiment, the sensor ultrasound waves may include longitudinal waves or shear waves. In an embodiment, the cavitation sensor includes cavitation sensor configured to determine a cavitation threshold in the mammal to the emitted ultrasonic shear waves.

In an embodiment, the system 100 includes a cavitation controller 144 configured to limit a power of the ultrasonic shear waves directed at the location to a level below a cavitation threshold of the mammal. In an embodiment, the cavitation controller is configured to limit a power of the ultrasonic shear waves directed at the location to a level below a cavitation threshold of the mammal in response to a detected cavitation event in the mammal. In an embodiment, the cavitation controller is configured to limit a power of the ultrasonic shear waves directed at the location to a level below a cavitation threshold of the mammal. in response to a detected cavitation event in the mammal to ultrasonic shear waves transmitted directed at the location. In an embodiment, the cavitation controller is configured to limit a power of the ultrasonic shear waves directed at the location to a level below a cavitation threshold of the mammal in response to a detected cavitation event in the mammal to ultrasonic shear waves directed at the location. In an embodiment, the cavitation controller is configured to limit the power level of the ultrasonic shear waves directed at the location to a level below a cavitation threshold by regulating a parameter of ultrasonic shear waves transmitted by the ultrasonic wave transmitter.

FIG. 1 illustrates an alternative embodiment of the system 105. The system includes the extracellular-fluid collection device 110 configured to be positioned at the location 184 on the skin 182 of the mammal 180. The system includes the ultrasonic wave transmitter 120 configured to emit ultrasonic shear waves 122 directable at the location. The ultrasonic shear waves have a frequency or amplitude selected to increase a permeability of the skin of the mammal to an extracellular-fluid. The system includes the sensor 132 configured to determine a rate or amount of extracellular-fluid collected by the extracellular-fluid collection device. The system includes the fluid collection controller 134 configured regulate a parameter of ultrasonic shear waves transmitted by the ultrasonic wave transmitter in response to the determined rate or amount of extracellular-fluid collected by the extracellular-fluid collection device. For example, fluid collection controller may be programed to increase an amplitude of the emitted ultrasonic shear waves if collection is too slow, decrease an amplitude of the emitted ultrasonic shear waves if collection is too fast, and stop the emitted ultrasonic shear waves if collection is completed.

In an embodiment, the ultrasonic shear waves 122 include shear waves having a frequency and amplitude selected to increase a permeability of the skin 182 of the mammal 180 to an extracellular-fluid. In an embodiment, the fluid collection controller 134 is configured to regulate an amplitude or frequency of the ultrasonic shear waves transmitted by the ultrasonic wave transmitter.

FIG. 1 illustrates another alternative embodiment of the system 105. The system includes the extracellular-fluid collection device 110 configured to be positioned at the location 184 on the skin 182 of the mammal 180. The system includes the ultrasonic wave transmitter 120 configured to emit ultrasonic shear waves 122 directable at the location. The ultrasonic shear waves have a frequency or amplitude selected to increase a permeability of the skin of the mammal to an extracellular-fluid. The system includes the cavitation sensor 142 configured to determine a cavitation event in the mammal responsive to the emitted ultrasonic shear waves. In an embodiment, the cavitation sensor is configured to determine a cavitation threshold in the mammal to the emitted ultrasonic shear waves. The system includes the cavitation controller 144 configured to limit a power of the ultrasonic shear waves directed at the location to a level below a cavitation threshold of the mammal in response to a detected cavitation event in the mammal. In an embodiment, the cavitation controller is configured to limit a power level of the ultrasonic shear waves directed at the location to a level below a cavitation threshold by regulating a parameter of ultrasonic shear waves transmitted by the ultrasonic wave transmitter.

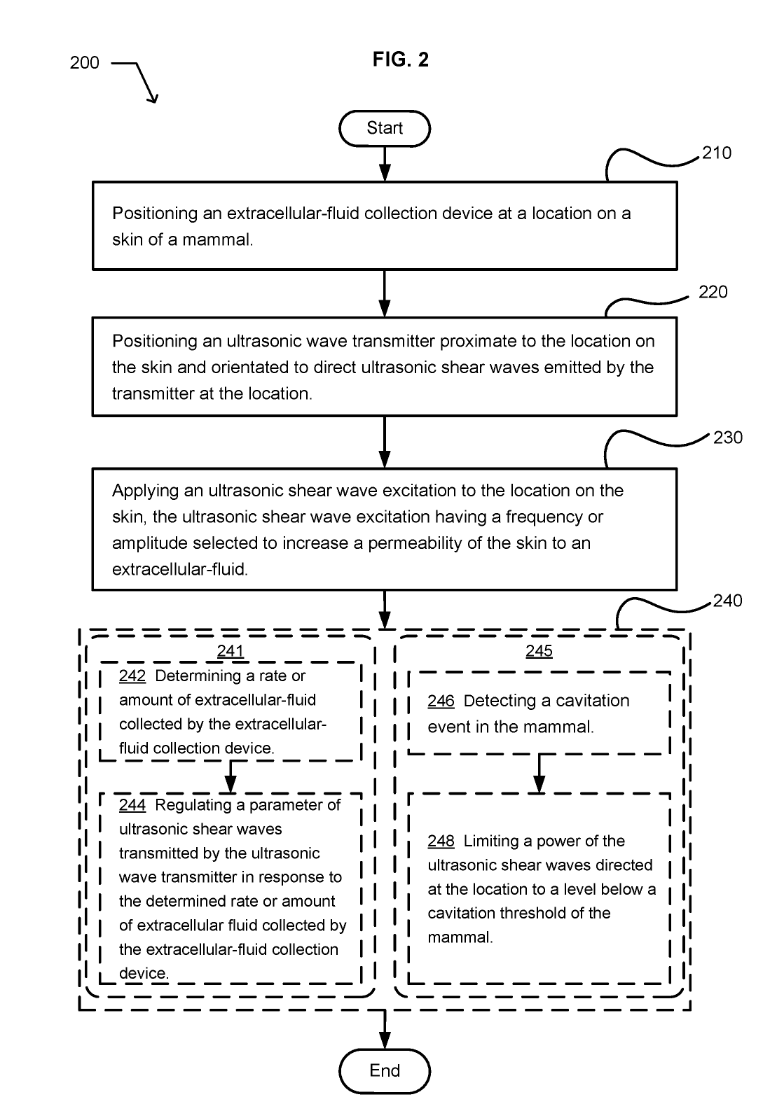

FIG. 2 illustrates an example operational flow 200 in which embodiments may be implemented. After a start operation, the operational flow includes a placement operation 210. The placement operation includes positioning an extracellular-fluid collection device at a location on a skin of a mammal. In an embodiment, the placement operation may be implemented using the extracellular-fluid collection device 110 described in conjunction with FIG. 1. An orientation operation 220 includes positioning an ultrasonic wave transmitter proximate to the location on the skin and orientated to direct ultrasonic shear waves emitted by the transmitter at the location. In an embodiment, the orientation operation may be implemented using the ultrasonic wave transmitter 120 described in conjunction with FIG. 1. A collection operation 230 includes applying an ultrasonic shear wave excitation to the location on the skin. The ultrasonic shear wave excitation has a frequency or amplitude selected to increase a permeability of the skin to an extracellular-fluid. In an embodiment, the collection operation may be implemented by turning on the ultrasonic wave transmitter 120 described in conjunction with FIG. 1. The operational flow includes an end operation.

In an embodiment of the placement operation 210, the extracellular-fluid collection device includes an extracellular-fluid collection device having a handheld form factor. In an embodiment of the placement operation, the extracellular-fluid collection device includes a removably attached extracellular-fluid collection device. In an embodiment of the orientation operation 220, the positioning includes positioning an ultrasonic wave transmitter proximate to the location on the skin and orientated to direct ultrasonic shear waves and ultrasonic longitudinal waves at the location.

In an embodiment of the collection operation 230, the applying includes applying an ultrasonic shear wave excitation to the location on the skin. The ultrasonic shear wave excitation has a frequency or amplitude selected to increase a movement of an analyte through the skin and into the fluid collection device. In an embodiment, the ultrasonic shear wave excitation has a frequency or amplitude selected to increase a movement of an analyte through the skin and into the fluid collection device without inducing a cavitation in tissue of the mammal. In an embodiment, the applying includes applying an ultrasonic shear wave excitation to the location on the skin. The ultrasonic shear wave excitation has a frequency or amplitude selected to transiently stretch the skin of the mammal. In an embodiment, the applying includes applying an ultrasonic shear wave excitation to the location on the skin. The ultrasonic shear wave excitation has a frequency or amplitude selected to increase a permeability of the skin to an extracellular-fluid without inducing a cavitation in the tissue of the mammal. In an embodiment, the applying includes applying a traveling ultrasonic shear wave excitation to the location on the skin. In an embodiment, the applying includes applying a surface ultrasonic shear wave excitation to the location on the skin. In an embodiment, the applying includes applying a two-dimensional surface shear wave excitation to the location on the skin. In an embodiment, the applying includes applying a three-dimensional shear wave excitation to the location on the skin. In an embodiment, the selected extracellular-fluid includes blood or a blood component. In an embodiment, the selected extracellular-fluid includes interstitial fluid. In an embodiment, the selected extracellular-fluid includes an exudate. In an embodiment, the selected extracellular-fluid includes a transudate.

In an embodiment, the operational flow 200 may include additional operations 240. Additional operations may include operations 241. The operations 241 include an operation 242 determining a rate or amount of extracellular-fluid collected by the extracellular-fluid collection device. The operation 241 includes an operation 244 regulating a parameter of ultrasonic shear waves transmitted by the ultrasonic wave transmitter in response to the determined rate or amount of extracellular fluid collected by the extracellular-fluid collection device. Additional operations may include operations 245. The operations 245 include an operation 246 detecting a cavitation event in the mammal. The operations 245 include limiting a power of the ultrasonic shear waves directed at the location to a level below a cavitation threshold of the mammal in response to a detected cavitation event in the mammal.

In an embodiment, the operational flow 200 includes creating a vacuum pressure between the extracellular-fluid collection device and the location on the skin. In an embodiment, the placement operation 210 includes positioning an extracellular-fluid collection device at the location on the skin of the mammal and creating a vacuum pressure between extracellular-fluid collection device and the location on the skin.

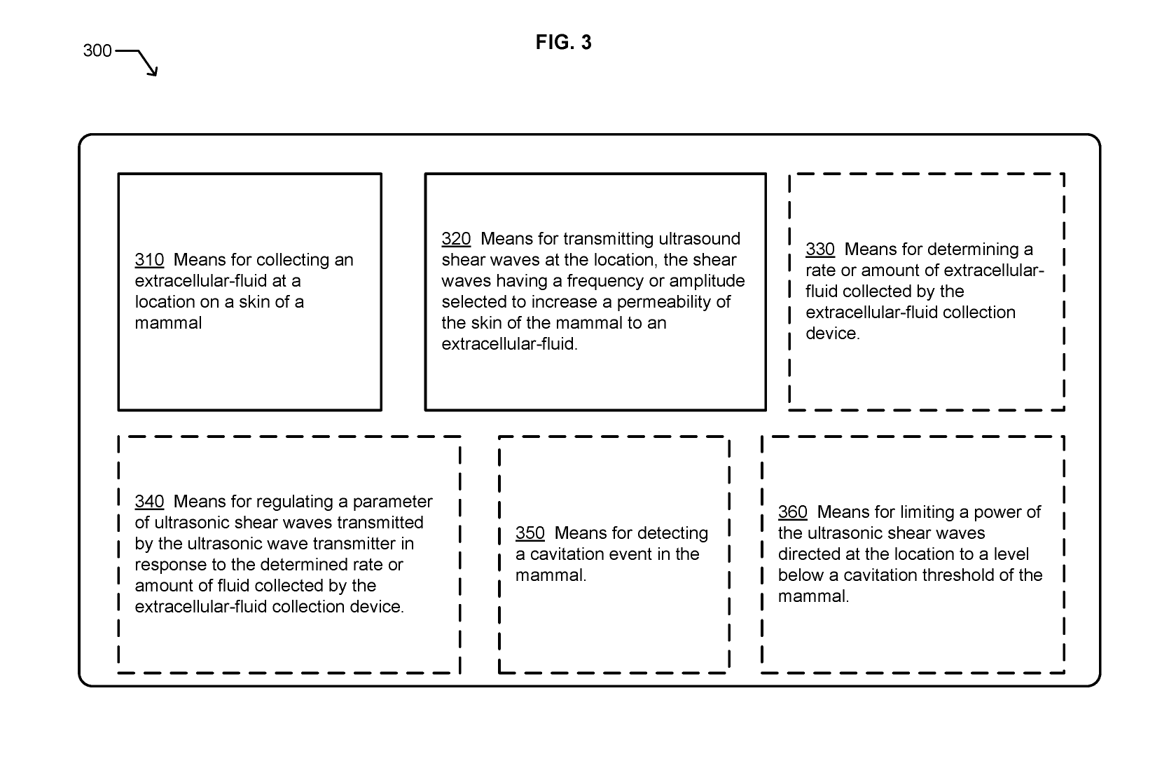

FIG. 3 illustrates an example system 300 in which embodiments may be implemented. The system includes means 310 for collecting an extracellular-fluid at a location on a skin of a mammal. The system includes means 320 for transmitting ultrasound shear waves at the location. The ultrasonic shear waves have a frequency or amplitude selected to increase a permeability of the skin of the mammal to an extracellular-fluid.

In an embodiment, the system 300 includes means 330 for determining a rate or amount of extracellular-fluid collected by the extracellular-fluid collection device. In an embodiment, the system includes means 340 for regulating a parameter of ultrasonic shear waves transmitted by the ultrasonic wave transmitter in response to the determined rate or amount of fluid collected by the extracellular-fluid collection device. In an embodiment, the system 300 includes means 350 for detecting a cavitation event in the mammal. In an embodiment, the system 300 includes means 360 for limiting a power of the ultrasonic shear waves directed at the location to a level below a cavitation threshold of the mammal in response to a detected cavitation event in the mammal.

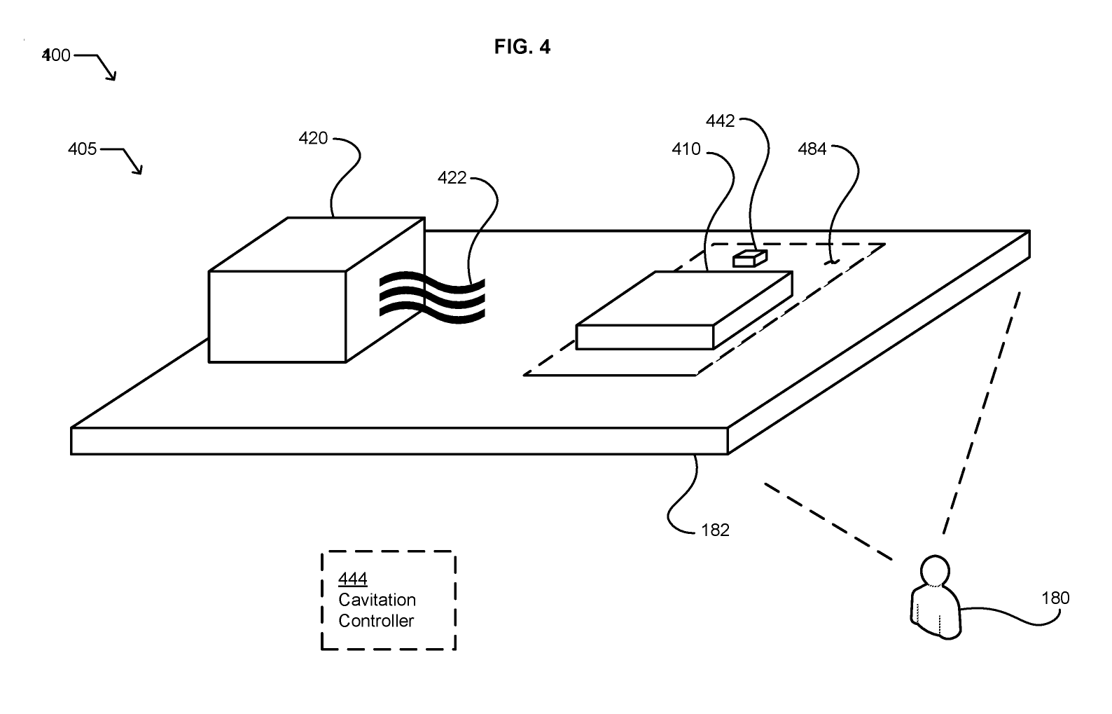

FIG. 4 illustrates an example environment 400 in which embodiments may be implemented. The environment includes the mammal 180 and a system 405. The system includes a medicament-eluting device 410 configured to be positioned at a location 484 on the skin 182 of the mammal. The system includes an ultrasonic wave transmitter 420 configured to emit ultrasonic shear waves 422 directable at the location. The ultrasonic shear waves have a frequency or amplitude selected to increase a permeability of the skin of the mammal to a medicament released by the medicament-eluting device. In an embodiment, the ultrasonic wave transmitter is configured to emit ultrasonic shear waves aimable or focusable at the location.

In an embodiment, the medicament-eluting device 420 is configured to be removably attached to the skin of the mammal. In an embodiment, the medicament-eluting device includes an ultrasound-transparent medicament-eluting device. For example, in an embodiment, the medicament-eluting device may have greater than a 90% transparency to the emitted ultrasonic shear waves 422. In an embodiment, the medicament-eluting device includes a transdermal patch. In an embodiment, the medicament-eluting device includes microprotrusions. In an embodiment, the microprotrusions include solid or hollow microneedles. For example, hollow microneedles may include a cannula with an approximate length of approximately 50-900 μm and an external diameter of not more than approximately 300 μm. For example, the microprotrusions may include an array of microneedles. In an embodiment, the microprotrusions may include microneedles, which may be fixed-state microneedles (e.g., fabricated from silicon, metals, or polymers), or biodegradable or dissolvable microneedles, (e.g., fabricated in hydrogel, polymers, or polysaccharides). In an embodiment, the microprotrusions may be hollow, e.g., in fluid communication with a reservoir holding a medicament; may be solid, e.g., coated with a medicament; or may encapsulate a medicament, e.g., in hydrogel or polysaccharides. Using microprotrusions with permeation enhancers such as ultrasound is expected to allow larger molecules to cross the skin 182. In an embodiment, the medicament-eluting device includes a strap or band configured to attach the medicament-eluting device to the mammal. In an embodiment, the medicament-eluting device includes a medicament-eluting device having a handheld form factor. In an embodiment, the medicament-eluting device includes an eluting chamber. In an embodiment, the medicament-eluting device includes a drug eluting device. In an embodiment, the medicament-eluting device includes a therapeutic substance eluting device. In an embodiment, the medicament-eluting device includes a transdermal patch. In an embodiment, the ultrasonic wave transmitter 420 includes an ultrasonic wave transmitter having a handheld form factor.

In an embodiment, the system 405 includes a structure carrying the medicament-eluting device 410 and the ultrasonic wave transmitter 420. In an embodiment, the system includes a cavitation sensor 442 configured to detect a cavitation event in the mammal 180. In an embodiment, the system includes a cavitation controller 444 configured to limit a power of the ultrasonic shear waves 422 directed at the location 484 to a level below a cavitation threshold of the mammal 180 in response to a detected cavitation event in the mammal. In an embodiment, the cavitation controller is configured to limit the power level of the ultrasonic shear waves directed at the location to a level below a cavitation threshold by regulating a parameter of ultrasonic shear waves transmitted by the ultrasonic wave transmitter.

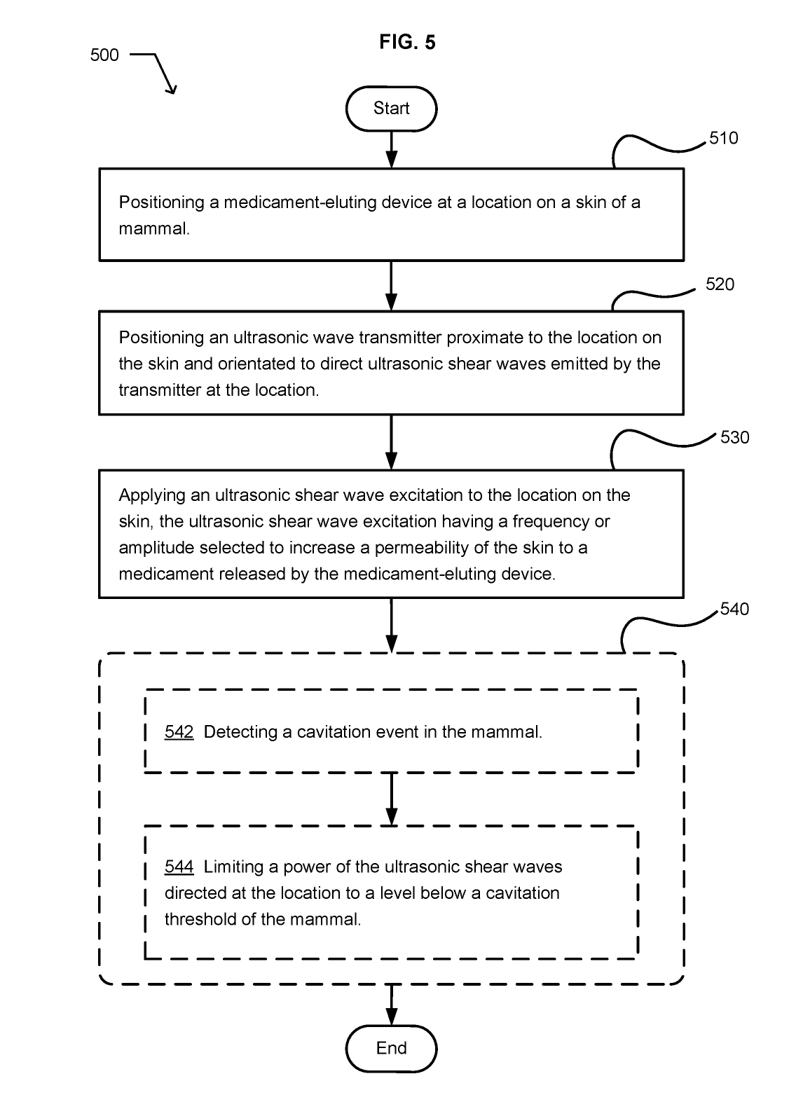

FIG. 5 illustrates an example operational flow 500 in which embodiments may be implemented. After a start operation, the operational flow includes a placement operation 510. The placement operation includes positioning a medicament-eluting device at a location on a skin of a mammal. In an embodiment, the placement operation may be implemented using the medicament-eluting device 410 described in conjunction with FIG. 4. An orientation operation 520 includes positioning an ultrasonic wave transmitter proximate to the location on the skin and orientated to direct ultrasonic shear waves emitted by the transmitter at the location. In an embodiment, the orientation operation may be implemented using the ultrasonic wave transmitter 420 described in conjunction with FIG. 4. A sonication operation 530 includes applying an ultrasonic shear wave excitation to the location on the skin. The ultrasonic shear wave excitation having a frequency or amplitude selected to increase a permeability of the skin to a medicament released by the medicament-eluting device. In an embodiment, the sonication operation includes applying an ultrasonic shear wave excitation to the location on the skin. The ultrasonic shear wave excitation having a frequency or amplitude selected to increase a permeability of the skin to a medicament released by the medicament-eluting device without inducing cavitation in tissue of the mammal. The operational flow includes an end operation.

In an embodiment of the orientation operation 520, the positioning includes positioning an ultrasonic wave transmitter proximate to the location on the skin and orientated to direct ultrasonic shear waves and ultrasonic longitudinal waves at the location. In an embodiment of the sonication operation 530, the applying includes applying an ultrasonic shear wave excitation to the location on the skin, the ultrasonic shear wave excitation having a frequency or amplitude selected to increase a movement of a medicament carried by the medicament-eluting device through the skin and into the mammal. In an embodiment of the sonication operation, the applying includes applying an ultrasonic shear wave excitation to the location on the skin, the ultrasonic shear wave excitation having a frequency or amplitude selected to transiently stretch the skin of the mammal.

In an embodiment, the operational flow 500 may include at least one additional operation. An additional operation 540 includes an operation 542 detecting a cavitation in the mammal. The additional operation includes an operation 544 limiting a power of the ultrasonic shear waves directed at the location to a level below a cavitation threshold of the mammal in response to a detected cavitation event in the mammal.

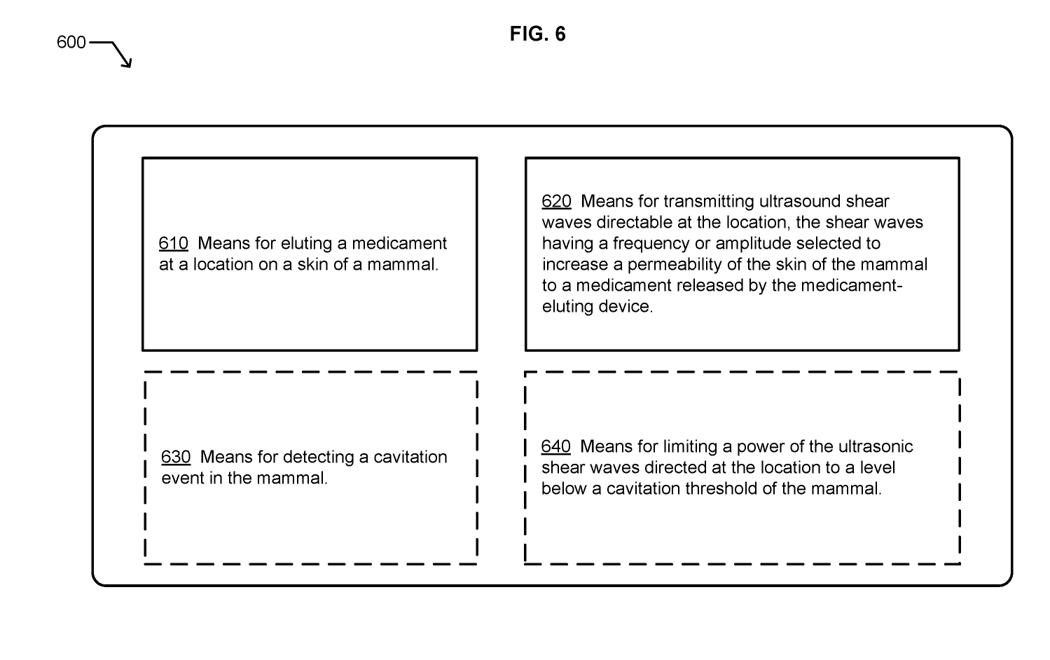

FIG. 6 illustrates an example system 600 in which embodiments may be implemented. The system includes means 610 for eluting a medicament at a location on a skin of a mammal. The system includes means 620 for transmitting ultrasound shear waves directable at the location. The ultrasonic shear waves have a frequency or amplitude selected to increase a permeability of the skin of the mammal to a medicament released by the medicament-eluting device.

In an embodiment, the system 600 includes means 630 for detecting a cavitation in the mammal. In an embodiment, the system includes means 640 for limiting a power of the ultrasonic shear waves directed at the location to a level below a cavitation threshold of the mammal in response to a detected cavitation event in the mammal. In an embodiment, the means for limiting a power includes means for limiting the power level of the ultrasonic shear waves directed at the location to a level below a cavitation threshold by regulating a parameter of ultrasonic shear waves transmitted by the ultrasonic wave transmitter.

All references cited herein are hereby incorporated by reference in their entirety or to the extent their subject matter is not otherwise inconsistent herewith.

In some embodiments, “configured” or “ configured to” includes at least one of designed, set up, shaped, implemented, constructed, or adapted for at least one of a particular purpose, application, or function. In some embodiments, “configured” or “configured to” includes positioned, oriented, or structured for at least one of a particular purpose, application, or function.

It will be understood that, in general, terms used herein, and especially in the appended claims, are generally intended as “open” terms. For example, the term “including” should be interpreted as “including but not limited to.” For example, the term “having” should be interpreted as “having at least.” For example, the term “has” should be interpreted as “having at least.” For example, the term “includes” should be interpreted as “includes but is not limited to,” etc. It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

In those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

With respect to the appended claims the recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Use of “Start,” “End,” “Stop,” or the like blocks in the block diagrams is not intended to indicate a limitation on the beginning or end of any operations or functions in the diagram. Such flowcharts or diagrams may be incorporated into other flowcharts or diagrams where additional functions are performed before or after the functions shown in the diagrams of this application. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system comprising:
an extracellular-fluid collection device configured to be positioned at a location on a skin of a mammal; and
an ultrasonic wave transmitter configured to emit ultrasonic shear waves directed at the location, the ultrasonic shear waves having a frequency or amplitude selected to increase a permeability of the skin of the mammal to an extracellular-fluid.

2. The system of claim 1, wherein the extracellular-fluid collection device is configured to be removably attached to the skin of the mammal.

3. The system of claim 1, wherein the extracellular-fluid collection device includes an ultrasound-transparent extracellular-fluid collection device.

4. The system of claim 1, wherein the extracellular-fluid collection device includes a transdermal patch.

5. The system of claim 1, wherein the extracellular-fluid collection device includes an absorbent extracellular-fluid collection device.

6. (canceled)

7. The system of claim 1, wherein the extracellular-fluid collection device includes an extracellular-fluid collection device having a handheld form factor.

8. The system of claim 1, wherein the extracellular-fluid collection device includes a collecting chamber.

9. The system of claim 1, wherein the ultrasonic wave transmitter is configured to emit traveling ultrasonic shear waves directed at the location on the skin.

10. The system of claim 1, wherein the ultrasonic wave transmitter is configured to emit ultrasonic shear waves focused at the location on the skin.

11. The system of claim 1, wherein the ultrasonic wave transmitter is configured to emit ultrasonic standing waves at the location on the skin.

12. The system of claim 1, wherein the ultrasonic wave transmitter is configured to emit both ultrasonic shear waves and longitudinal waves directed at the location on the skin.

13. The system of claim 1, wherein the ultrasonic wave transmitter is configured to emit surface shear waves.

14. The system of claim 1, wherein the ultrasonic wave transmitter is configured to emit two-dimensional surface shear waves

15. (canceled)

16. The system of claim 1, wherein the ultrasonic shear waves have a frequency and amplitude selected to increase a permeability of the skin of the mammal to an extracellular-fluid.

17. The system of claim 1, wherein the ultrasonic shear waves have a frequency greater than 1 MHz.

18. (canceled)

19. The system of claim 1, wherein the ultrasonic shear waves have a frequency greater than 10 MHz.

20. (canceled)

21. The system of claim 1, wherein the ultrasonic wave transmitter includes a handheld form factor.

22. The system of claim 1, wherein the ultrasonic wave transmitter is configured to be removeably attached to the skin.

23. The system of claim 1, further comprising:
a housing carrying the extracellular-fluid collection device and the ultrasonic wave transmitter.

24. The system of claim 1, further comprising:
a sensor configured to determine a rate or amount of extracellular-fluid collected by the extracellular-fluid collection device.

25. The system of claim 1, further comprising:
a fluid collection controller configured to regulate a parameter of ultrasonic shear waves transmitted by the ultrasonic wave transmitter in response to a determined rate or amount of fluid collected by the extracellular-fluid collection device.

26. The system of claim 1, further comprising:
a cavitation sensor configured to detect a cavitation event in the mammal.

27. (canceled)

28. (canceled)

29. (canceled)

30. (canceled)

31. The system of claim 1, further comprising:
a cavitation controller configured to limit a power of the ultrasonic shear waves directed at the location to a level below a cavitation threshold of the mammal.

32. (canceled)

33. (canceled)

34. A system comprising:
an extracellular-fluid collection device configured to be positioned at a location on a skin of a mammal;
an ultrasonic wave transmitter configured to emit ultrasonic shear waves directed at the location, the ultrasonic shear waves having a frequency or amplitude selected to increase a permeability of the skin of the mammal to an extracellular-fluid;
a sensor configured to determine a rate or amount of extracellular-fluid collected by the extracellular-fluid collection device; and
a fluid collection controller configured regulate a parameter of ultrasonic shear waves transmitted by the ultrasonic wave transmitter in response to the determined rate or amount of extracellular-fluid collected by the extracellular-fluid collection device.

35. (canceled)

36. The system of claim 34, wherein the fluid collection controller is configured to regulate an amplitude or frequency of the ultrasonic shear waves transmitted by the ultrasonic wave transmitter.

37. A system comprising:
an extracellular-fluid collection device configured to be positioned at a location on a skin of a mammal;
an ultrasonic wave transmitter configured to emit ultrasonic shear waves directed at the location, the ultrasonic shear waves having a frequency or amplitude selected to increase a permeability of the skin of the mammal to an extracellular-fluid;
a cavitation sensor configured to determine a cavitation event in the mammal responsive to the emitted ultrasonic shear waves; and
a cavitation controller configured to limit a power of the ultrasonic shear waves directed at the location to a level below a cavitation threshold of the mammal.

38. (canceled)

39. A method comprising:
positioning an extracellular-fluid collection device at a location on a skin of a mammal;
positioning an ultrasonic wave transmitter proximate to the location on the skin and orientated to direct ultrasonic shear waves emitted by the transmitter at the location;
applying an ultrasonic shear wave excitation to the location on the skin, the ultrasonic shear wave excitation having a frequency or amplitude selected to increase a permeability of the skin to an extracellular-fluid.

40. (canceled)

41. (canceled)

42. The method of claim 39, wherein the positioning includes positioning an ultrasonic wave transmitter proximate to the location on the skin and orientated to direct ultrasonic shear waves and ultrasonic longitudinal waves at the location.

43. (canceled)

44. (canceled)

45. The method of claim 39, wherein the applying includes applying an ultrasonic shear wave excitation to the location on the skin, the ultrasonic shear wave excitation having a frequency or amplitude selected to increase a permeability of the skin to an extracellular-fluid without inducing a cavitation in the tissue of the mammal.

46. The method of claim 39, wherein the applying includes applying a traveling ultrasonic shear wave excitation to the location on the skin.

47. The method of claim 39, wherein the applying includes applying a surface ultrasonic shear wave excitation to the location on the skin.

48. The method of claim 39, wherein the applying includes applying a two-dimensional surface ultrasonic shear wave excitation to the location on the skin.

49. (canceled)

50. (canceled)

51. (canceled)

52. (canceled)

53. The method of claim 39, further comprising:
regulating a parameter of ultrasonic shear waves transmitted by the ultrasonic wave transmitter in response to a determined rate or amount of extracellular fluid collected by the extracellular-fluid collection device.

54. (canceled)

55. The method of claim 1, further comprising:
limiting a power of the ultrasonic shear waves directed at the location to a level below a detected cavitation threshold of the mammal.

56. The method of claim 39, further comprising:
creating a vacuum between extracellular-fluid collection device and the location on the skin.

57. The method of claim 39, wherein the positioning includes positioning an extracellular-fluid collection device at the location on the skin of the mammal and creating a vacuum pressure between extracellular-fluid collection device and the location on the skin.

58-62 (canceled)
